(19) United States
(12) Patent Application Publication
(10) Pub. No.: US 2001/0016571 A1
(43) Pub. Date: Aug. 23, 2001

(54) N-[ (R) -1-{3- (4- piperidyl) propionyl } -3-piperidylcarbonyl] -2 (S) -acetylamino-beta -alanine as fibrinogen receptor antagonist

(75) Inventors: Mitsuru Ohkubo, Hyogo (JP); Fumie Takahashi, Osaka (JP); Toshio Yamanaka, Osaka (JP); Masayuki Kato, Kyoto (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: FUJISAWA PHARMACEUTICAL CO., LTD., OSAKA (JP)

(21) Appl. No.: 09/805,996

(22) Filed: Mar. 15, 2001

Related U.S. Application Data
(63) Continuation of application No. 09/147,011, filed on Sep. 11, 1998, now abandoned.
(63) Continuation of application No. PCT/JP97/00769, filed on Mar. 12, 1997.

(30) Foreign Application Priority Data
Mar. 13, 1996 (ZA) 96/2033

(51) Int. Cl. A61K 38/05; C07D 211/32

(52) U.S. Cl. 514/19; 546/189

(57) ABSTRACT

This invention relates to β-alanine derivative represented by the following formula (I);

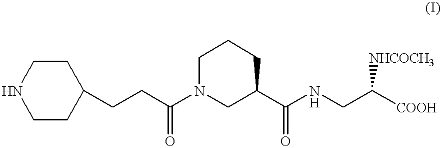

which is glycoprotein IIB/IIla antagonist, inhibitor of blood platelets aggregation and inhibitor of the binding of fibrinogen to blood platelets, to processes for the preparation thereof, to a pharmaceutical composition comprising the same and to a method for the prevention and/or treatment diseases indicated in the specification to a human being or an animal.

### TECHNICAL FIELD

The present invention relates to β-alanine derivative. More particularly, it relates to β-alanine derivative which is glycoprotein IIb/IIIa antagonist, inhibitor of blood platelets aggregation and inhibitor of the binding of fibrinogen to blood platelets.

### BACKGROUND ART

In European Patent Application No. 512,831 A1, there are disclosed fibrinogen receptor antagonists.

In European Patent Application No. 445,796 A2, there are disclosed inhibitor of blood platelets aggregation.

### DISCLOSURE OF INVENTION

The present invention relates to β-alanine derivative. More particularly, it relates to β-alanine derivative which is glycoprotein IIb/IIIa antagonist and inhibitor of platelet aggregation, and useful as:

a drug for the prevention and/or the treatment of diseases caused by thrombus formation such as arterial thrombosis; arterial sclerosis; ischemic heart diseases [e.g. angina pectoris (e.g. stable angina pectoris, unstable angina pectoris including imminent infarction, etc.), myocardial infarction (e.g. acute myocardial infarction, etc.), coronary thrombosis, etc.]; ischemic brain diseases [e.g. cerebral infarction {e.g. cerebral thrombosis (e.g. acute cerebral thrombosis, etc.), cerebral embolism, etc.}, transient cerebral ischemia (e.g. transient ischemic attack, etc.), cerebrovascular spasm after cerebral hemorrhage (e.g. cerebrovascular spasm after subarachnoid hemorrhage, etc.), etc.]; pulmonary vascular diseases (e.g. pulmonary thrombosis, pulmonary embolism etc.); peripheral circulatory disorder [e.g. arteriosclerosis obliterans, thromboangiitis obliterans (i.e. Burger's disease), Raynaud's disease, complication of diabetes mellitus (e.g. diabetic angiopathy, diabetic neuropathy, etc.), phlebothrombosis (e.g. deep vein thrombosis, etc.), etc.] or the like;

a drug for the prevention and/or the treatment of restenosis and/or reocclusion such as restenosis and/or reocclusion after percutaneous transluminal coronary angioplasty (PTCA), restenosis and/or reocclusion after the administration of thrombolytic drug (e.g. tissue plasminogen activator (TPA), etc.) or the like;

a drug for the adjuvant therapy with thrombolytic drug (e.g. TPA, etc.) or anticoagulant (e.g. heparin, etc.);

a drug for the prevention and/or the treatment of the thrombus formation in case of vascular surgery, valve replacement, extracorporeal circulation [e.g. surgery (e.g. open heart surgery, pump-oxygenator, etc.) hemodialysis, etc.], transplantation, or the like;

a drug for the prevention and/or he treatment of disseminated intravascular coagulation (DIC), thrombotic thrombocycopenia, essential thrombocytosis, Inflammation (e.g. nephritis, etc.), immune diseases, or the like;

a drug for inhibiting of metastasis; or the like.

The β-alanine derivative of the present invention is expected to be useful as an inhibitor of cell adhesion and so is expected to be useful as

a drug for the prevention and/or the treatment of disseminated intravascular coagulation (DIC), thrombotic thrombocytopenia, essential thrombocytosis, inflammation (e.g. nephritis, etc.), immune diseases, or the like;

a drug for inhibiting of metastasis; or the like.

The object β-alanine derivative of the present invention can be shown by the following formula (I):

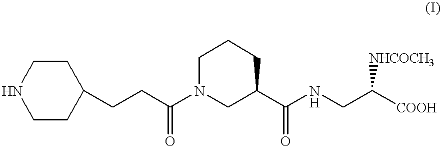

The object compound (I) can be prepared by the following processes.

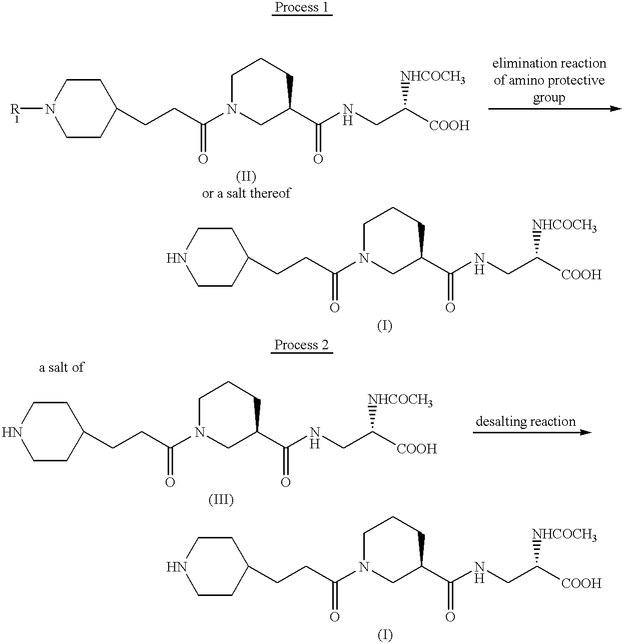

wherein R¹ is amino protective group.

Suitable salt of the compounds (II) and (III) are pharmaceutically acceptable salt such as conventional non-toxic salt and include a metal salt such as an alkali metal salt [e.g. sodium salt, potassium salt, etc.] and an alkaline earth metal salt [e.g. calcium salt, magnesium salt, etc.] an ammonium salt, an organic base salt [e.g. trimethylamine salt, triethylamine salt, pyridine salt, picoline salt dicyclohexylamine salt, N,N-dibenzylethylenediamine salt, etc.], an organic acid addition salt [e.g. formate, acetate, trifluoroacetate, maleate, tartrate, methanesulfonate, benzenesulfonate, toluenesulfonate, etc.], an inorganic acid addition salt [e.g. hydrochloride, hydrobromide, hydroiodide, sulfate, phosphate, etc.], a salt with an amino acid [e.g. arginine salt, aspartic acid salt, glutamic acid salt, etc.] and the like.

In the above and subsequent descriptions of this specification, suitable examples of the various definitions are explained in detail as follows:

The term “lower” is intended to mean 1 to 6 carbon atom(s), unless otherwise indicated.

The term “higher” is used to intend a group having 7 to 20 carbon atoms, unless otherwise provided.

Suitable “lower alkyl” may be straight or branched ones such as methyl, ethyl, isopropyl, propyl, butyl, isobutyl, sec-butyl, t-butyl, pentyl, isopentyl, hexyl, isohexyl or the like.

Suitable “amino protective groups” may include acyl group as explained below, a conventional protecting group such as ar(lower)alkyl which may have 1 to 3 suitable substituent(s) (e.g. benzyl, phenethyl, 1-phenylethyl, benzhydryl, trityl, etc.), [5-(lower)alkyl-2-oxo-1,3-dioxol-4-yl](lower)alkyl [e.g. (5-methyl-2-oxo-1,3-dioxol-4-yl)methyl, etc.] or the like; and the like.

Suitable “acyl group” and “acyl” may include aliphatic acyl, aromatic acyl, arylaliphatic acyl and heterocyclic-aliphatic acyl derived from carboxylic acid, carbonic acid, carbamic acid, sulfonic acid, and the like.

Suitable example of said “acyl group” may be illustrated as follows:

aliphatic acyl such as lower or higher alkanoyl (e.g., formyl, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl, icosanoyl, etc.);

lower or higher alkoxycarbonyl (e.g., methoxycarbonyl, ethoxycarbonyl, t-butoxycarbonyl, t-pentyloxycarbonyl, heptyloxycarbonyl, etc.);

lower or higher alkylsulfonyl (e.g., methylsulfonyl, ethylsulfonyl, etc.);

lower or higher alkoxysulfonyl (e.g., methoxysulfonyl, ethoxysulfonyl, etc.); or the like;

aromatic acyl such as

aroyl (e.g., benzoyl, toluoyl, naphthoyl, etc.); ar(lower)alkanoyl [e.g., phenyl(C₁-C₆)alkanoyl (e.g., phenylacetyl, phenylpropanoyl, phenylbutanoyl, phenylisobutanoyl, phenylpentanoyl, phenylhexanoyl, etc.), naphthyl(C₁-C₆)alkanoyl (e.g., naphthylacetyl, naphthylpropanoyl, naphthylbutanoyl, etc.), etc.];

ar(lower)alkenoyl [e.g., phenyl(C₃-C₆)alkenoyl (e.g., phenylpropenoyl, phenylbutenoyl, phenylmethacryloyl, phenylpentenoyl, phenylhexencyl, etc.), naphthyl (C₃-C₆)-alkenoyl (e.g., naphthylpropenoyl, naphthylbutenoyl, etc.), etc.];

ar(lower)alkoxycarbonyl [e.g., phenyl (C₁-C₆)-alkoxycarbonyl (e.g., benzyloxycarbonyl, etc.), etc.];

aryloxycarbonyl (e.g., phenoxycarbonyl, naphthyloxycarbonyl, etc.);

aryloxy(lower)alkanoyl (e.g., phenoxyacetyl, phenoxypropionyl, etc.);

arylcarbamoyl (e.g., phenylcarbamoyl, etc.);

arylthiocarbamoyl (e.g., phenylthiocarbamoyl, etc.); arylglyoxyloyl (e.g., phenylglyoxyloyl, naphthylglyoxyloyl, etc.);

arylsulfonyl which may have 1 to 4 lower alkyl (e.g., phenylsulfonyl, p-tolylsulfonyl, etc.); or the like;

heterocyclic acyl such as

heterocycliccarbonyl;

heterocyclic(lower)alkanoyl (e.g., heterocyclicacetyl, heterocyclicpropanoyl, heterocyclicbutanoyl, heterocyclicpentanoyl, heterocyclichexanoyl, etc.);

heterocyclic(lower)alkenoyl (e.g., heterocyclicpropenoyl, heterocyclicbutenoyl, heterocyclicpentenoyl, heterocyclichexenoyl, etc.);

heterocyclicglyoxyloyl; or the like; and the like.

Suitable “heterocyclic moiety” in the terms “heterocycilccarbonyl”, “heterocyclic (lower)alkyl”, “heterocyclic(lower)alkenoyl” and “heterocyclicglyoxyloyl” as mentioned above, and “heterocyclic group” mean saturated or unsaturated monocyclic or polycyclic heterocyclic group containing at least one hetero-atom such as an oxygen, sulfur, nitrogen atom and the like, in which the preferable heterocyclic group may be heterocyclic group such as

unsaturated 3 to 8-membered (more preferably 5 or 6-membered) heteromonocyclic group containing 1 to 4 nitrogen atom(s), for example, pyrrolyl, pyrrolinyl, imidazolyl, pyrazolyl, pyridyl, dihydropyridyl, pyrimidyl, pyrazinyl, pyridazinyl, triazolyl (e.g., 4H-1,2,4-triazolyl, 1H-1,2,3-triazolyl, 2H-1,2,3-triazcoyl, etc.), tetrazolyl (e.g., 1H-tetrazolyl, 2H-tetrazolyl, etc.), etc.;

saturated 3 to 8-membered (more preferably 5 or 6-membered) heteromonocyclic group containing 1 to 4 nitrogen atom(s), for example, pyrrolidinyl, imidazolidinyl, piperidyl, piperazinyl, etc.;

unsaturated condensed heterocyclic group containing 1 to 4 nitrogen atom(s), for example, indolyl, isoindolyl, indolinyl, indolizinyl, benzimidazolyl, quinolyl, dihydroquinolyl, isoquinolyl, indazolyl, quinoxalinyl, dihydroquinoxalinyl, benzotriazolyl, etc.;

unsaturated 3 to 8-membered (more preferably 5 or 6-membered) heteromonocyclic group containing 1 to 2 oxygen atom(s) and 1 to 3 nitrogen atom(s), for example, oxazolyl, isoxazolyl, oxadiazolyl (e.g., 1,2,4-oxadiazolyl, 1,3,4-oxadiazolyl, 1,2,5-oxadiazolyl, etc.), etc.;

saturated 3 to 8-membered (more preferably 5 or 6-membered) heteromonocyclic group containing 1 to 2 oxygen atom(s) and 1 to 3 nitrogen atom(s), for example, morpholinyl, sydnonyl, etc.;

unsaturated condensed heterocyclic group containing 1 to 2 oxygen atom(s) and 1 to 3 nitrogen atom(s), for example, benzoxazolyl, benzoxadlazolyl, etc.;

unsaturated 3 to 8-membered (more preferably 5 or 6-membered) heteromonocyclic group containing 1 to 2 sulfur atom(s) and 1 to 3 nitrogen atom(s), for example, thiazolyl, isothiazolyl, thiadiazolyl (e.g., 1,2,3-thiadiazolyl, 1,2,4-thiadiazolyl, 1,3,4-thiadiazolyl, 1,2,5-thiadiazolyl, etc.), dihydrothiazinyl, etc.;

saturated 3 to 8-membered (more preferably 5 or 6-membered) heteromonocyclic group containing 1 to 2 sulfur atom(s) and 1 to 3 nitrogen atom(s), for example, thiazolidinyl, etc.;

unsaturated 3 to 8-membered (more preferably 5 or 6-membered) heteromonocyclic group containing 1 to 2 sulfur atom(s), for example, thienyl, dihydrodithiinyl, dihydrodithionyl, etc.;

unsaturated condensed heterocyclic group containing 1 to 2 sulfur atom(s) and 1 to 3 nitrogen atom(s), for example, benzothiazolyl, benzothiadiazolyl, etc.;

unsaturated 3 to 8-membered (more preferably 5 or 6-membered) heteromonocyclic group containing an oxygen atom, for example, furyl, etc.;

unsaturated 3 to 8-membered (more preferably 5 or 6-membered) heteromonocyclic group containing an oxygen atom and 1 to 2 sulfur atom(s), for example, dihydrooxathiinyl, etc.;

unsaturated condensed heterocyclic group containing 1 to 2 sulfur atom(s), for example, benzothienyl, benzodithiinyl, etc.;

unsaturated condensed heterocyclic group containing an oxygen atom and 1 to 2 sulfur atom(s), for example, benzoxathiinyl, etc.; and the like.

The acyl moiety as mentioned above may have one to ten, same or different, suitable substituent(s) such as

lower alkyl (e.g., methyl, ethyl, propyl, etc.);

lower alkoxy (e.g., methoxy, ethoxy, propoxy, etc.);

lower alkylthio (e.g., methylthio, ethylthio, etc.);

lower alkylamino (e.g., methylamino, ethylamino, propylamino, etc.);

cyclo(lower)alkyl [e.g. cyclo(C₃-C₆)alkyl (e.g., cyclopentyl, cyclohexyl, etc.]);

cyclo(lower)alkenyl [e.g. cyclo(C₃-C₆)alkenyl (e.g., cyclohexenyl, cyclohexadienyl, etc.);

halogen (e.g., fluorine, chlorine, bromine, iodine); amino; amino protective group as mentioned above; hydroxy; protected hydroxy as mentioned below; cyano; nitro; carboxy; protected carboxy; sulfo; sulfamoyl; imino; oxo;

amino(lower)alkyl (e.g., aminomethyl, aminoethyl, etc.); carbamoyloxy; hydroxy(lower)alkyl (e.g., hydroxymethyl, 1 or 2-hydroxyethyl, 1 or 2 or 3-hydroxypropyl, etc.), or the like.

Suitable “protected hydroxy” may include acyl as mentioned above, phenyl(lower)aikyl which may have one or more suitable substituent(s) (e.g., benzyl, 4-methoxybenzyl, trityl, etc.), trisubstituted silyl [e.g., tri(lower)alkylsilyl (e.g., trimethylsilyl, t-butyldimethylsilysl, etc.), etc.], tetrahydropyranyl and the like.

The more preferred example of “amino protective group” may be lower alkoxycarbonyl or ar(lower)alkoxycarbonyl and the most preferred one may be t-butoxycarbonyl or benzyloxycarbonyl.

### Process 1

The object compound (I) can be prepared by subjecting a compound (II) or a salt thereof to elimination reaction of amino protective group.

This reaction is carried out in accordance with a conventional method such as hydrolysis, reduction or the like.

The hydrolysis is preferably carried out in the presence of a base or an acid including Lewis acid.

Suitable base may include an inorganic base and an organic base such as an alkali metal [e.g. sodium, potassium, etc.], an alkaline earth metal [e.g. magnesium, calcium, etc.], the hydroxide or carbonate or bicarbonate thereof, trialkylamine [e.g. trimethylamine, triethylamine, etc.], picoline, 1,5-diazabicyclo[4.3.0]non-5-ene, 1,4-diazabicyclo[2.2.2]octane, 1,8-diazabicyclo[5.4.0]undec-7-ene, or the like.

Suitable acid may include an organic acid [e.g. formic acid, acetic acid, propionic acid, trichloroacetic acid, trifluoroacetic acid, etc.] and an inorganic acid [e.g. hydrochloric acid, hydrobromic acid, sulfuric acid, hydrogen chloride, hydrogen bromide, etc.].

The elimination using Lewis acid such as trihaloacetic acid [e.g. trichloroacetic acid, trifluoroacetic acid, etc.] or the like is preferably carried out in the presence of cation trapping agents [e.g. anisole, phenol, etc.].

The reaction is usually carried cut in a solvent such as water, an alcohol [e.g. methanol, ethanol, etc.], methylene chloride, tetrahydrofuran, a mixture thereof or any other solvent which does not adversely influence the reaction. A liquid base or acid can be also used as the solvent. The reaction temperature is not critical and the reaction is usually carried out under cooling to warming.

The reduction method applicable for the elimination reaction may include chemical reduction and catalytic reduction are a combination of metal [e.g. tin, zinc, iron, etc.] or metallic compound [e.g. chromium chloride, chromium acetate, etc.] and an organic or inorganic acid [e.g. formic acid, acetic acid, pronionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g. platinum plate, spongy platinum, platinum black, colloidal platinum, platinum oxide, platinum wire, etc.], palladium catalysts [e.g. spongy palladium, palladium black, palladium oxide, palladium on carbon, colloidal palladium, palladium on barium, sulfate, palladium on barium carbonate, etc.], nickel catalysts [e.g. reduced nickel, nickel oxide, Raney nickel, etc.], cobalt catalysts [e.g. reduced cobalt, Raney cobalt, etc.], iron catalysts [e.g. reduced iron, Raney iron, etc.], copper catalysts [e.g. reduced copper, Raney copper, Ullman copper, etc.] and the like.

The reduction is usually carried out in a conventional solvent which does not adversely influence the reaction such as water, methanol, ethanol, propanol, N,N-dimethylformamide, or a mixture thereof. Additionally, in case that the above-mentioned acids to be used in chemical reduction are in liquid, they can also be used as a solvent. Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to warming.

When the object compound (I) thus obtained is in a salt form, it can be converted into a free form in a conventional manner.

### Process 2

The object compound (I) can be prepared by subjecting a compound (III) to desalting reaction.

This reaction is carried out in accordance with a conventional method such as neutralization, recrystallization, desalting resin column chromatography, or the like.

The compounds obtained by the above Processes 1 and 2 can be isolated and purified by a conventional method such as pulverization, recrystallization, column-chromatography, reprecipitation, or the like.

The object compound (I) may include solvated compound [e.g., enclosure compound (e.g., hydrate, etc.)].

The object compound (I) is crystalline, and so it is stable and easy to handle.

Now in order to show the utility of the object compound (I), some pharmacological test data of the compound (I) of the present invention are shown in the following.

Test 1: Fibrinogen Binding to Platelets

Test Compound

the compound of Example 1

Test Method

Washed human platelets were prepared from platelet-rich plasma by gel filtration. The washed platelets were activated with 20 μM ADP for 10 minutes and then fixed for 30 minutes with 0.8% paraformaldehyde. The platelets were then washed by centrifugation and suspended in HEPES-Tyrodes buffer containing 2 nM CaCl₂ and 1 nM MgCl₂.

350 μl of platelets were incubated at a final concentration of 2×10⁸/ml with 10 μg/ml FITC-fibrinogen and test compound. The reaction was left for 30 minutes at room temperature. The FITC fluoresence of bound fibrinogen was measured by using a FACS can flow cytometer.

Specific binding was calculated by subtracting the binding in the presence of 50-fold excess unlabeled fibrinogen. Result was expressed at IC₅₀ value, i.e. dose required to inhibit the binding by 50%.

Test Result

Test Compound IC₅₀

(1) 1.6×10⁻⁸

The pharmaceutical composition of the present invention can be used in the form of a pharmaceutical preparation, for example, in solid, semisolid or liquid form, which contains the object compound (I), as an active ingredient in admixture with an organic or inorganic carrier or excipient suitable for rectal, pulmonary (nasal or buccal inhalation), nasal, ocular, external (topical), oral or parenteral (including subcutaneous, intravenous and intramuscular) administrations or insufflation.

The active ingredient may be compounded, for example, with the usual non-toxic, pharmaceutically acceptable carriers for tablets, pellets, troches, capsules, suppositories, creams, ointments, aerosols, powders for insufflation, solutions, emulsions, suspensions, and any other form suitable for use. And, if necessary, in addition, auxiliary, stabilizing, thickening and coloring agents and perfumes may be used.

The object compound (I) is included in the pharmaceutical composition in an amount sufficient to produce the desired effect upon the process or condition of the diseases.

The pharmaceutical composition of the present invention can be manufactured by the conventional method in this field of the art. If necessary, the technique generally used in this field of the art for improving the bioavailability of a drug can be applied to the pharmaceutical composition of the present invention.

For applying the composition to a human being or an animal, it is preferable to apply it by intravenous (including i.v. infusion), intramuscular, pulmonary, or oral administration, or insufflation including aerosols from metered dose inhalator, nebulizer or dry powder inhalator.

While the dosage of therapeutically effective amount of the object compound (I) varies from and also depends upon the age and condition of each individual patient to be treated, in the case of intravenous administration, a daily dose of 0.001-100 mg of the object compound (I) per kg weight of a human being or an animal, in the case of intramuscular administration, a daily dose of 0.001-100 mg of the object compound (I) per kg weight of a human being or an animal, in case of oral administration, a daily dose of 0.001-200 mg of the object compound (I) per kg weight of a human being or an animal in generally given for the prevention and/or the treatment of aforesaid diseases in a human being or an animal.

The following Example is given for the purpose of illustrating the present invention in more detail.

### EXAMPLE 1

A mixture of N-[(R)-1-{3-(1-benzyloxycarbonyl-4-piperidyl)propionyl}-3-piperidylcarbonyl]-2(S)-acetylamino-β-alanine (0.5 g) 1N HCl (0.94 ml) and 10% Pd-C (0.1 g) in tetrahydrofuran (5 ml) was hydrogenated at atmospheric pressure for 2 hours. After the catalyst was removed by filtration, the filtrate was concentrated in vacuo. The residue was resolved in water, and neutralized with saturated aqueous NaHCO₃, desalted by using the resin of HP-20 eluting with isopropanol:H₂O=(1:1), then freeze-dried and recrystallized from ethanol to give N-[(R)-1-{3-(4-piperidyl)propionyl-3-piperidylcarbonyl]-2(S)-acetylamino-β-alanine (0.34 g, 91.0%) as white crystal.

IR (KBr pellet) : 2943, 2862, 1608 cm⁻¹

NMR (D₂O, δ) : 1.31-1.88 (8H, m), 1.94-2.03 (4H, m), 2.03 (3H, s), 2.39-2.54 (3H, m), 2.80-3.05 (3H, m) , 3.19-3.48 (5H, m), 3.63-3.74 (1H, m), 3.81-3.95 (1H, m) 4.18-4.34 (1H, m), 4.35-4.41 (1H, m)

Elemental Analysis Calcd. for C₁₉H₃₂N₄O₅.1.6H₂O: C 53.66, H 8.34, N 13.17 Found : C 53.63, H 8.56, N 13.03

1. A compound of the formula (I):

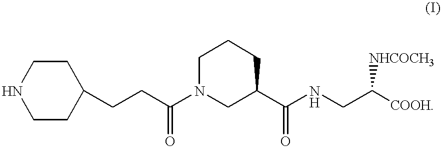

2. A process for preparing a compound of claim 1, which comprises
(i) subjecting a compound of the formula: 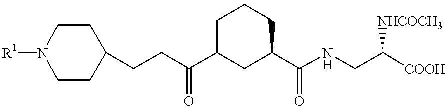
wherein R¹ is amino protective group, or a salt thereof, to elimination reaction of the amino protective group, to give a compound of the formula: 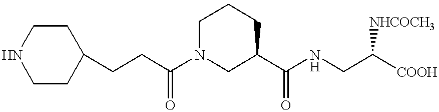
(ii) subjecting a salt of a compound of the formula: 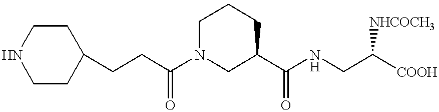
to desalting reaction, to give a compound of the formula:

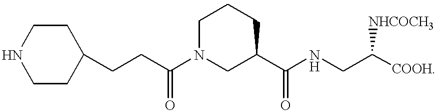

3. A pharmaceutical composition which comprises, as an active ingredient, a compound of claim 1 in admixture with pharmaceutically acceptable carriers or excipients.

4. Use of a compound of claim 1 for the manufacture of a medicament.

5. A compound of claim 1 for use as a medicament.

6. A method for the prevention and/or the treatment of diseases caused by thrombus formation; estenosis or reocclusion; the thrombus formation in case of vascular surgery, valve replacement, extracorporeal circulation or transplantation; disseminated intravascular coagulation; thrombotic thrombocytopenic; essential thrombocytosis; inflammation; immune diseases; or metastasis; or for the adjuvant therapy with thrombolytic drug or anticoagulant; which comprises administering a compound of claim 1 to a human being or an animal.
